(19) United States
(12) Patent Application Publication
(10) Pub. No.: US 2007/0141140 A1
(43) Pub. Date: Jun. 21, 2007

(54) Semi-solid formulations for immediate release intended for the oral administration of drugs

(75) Inventor: Alessandro Martini, (US)

Correspondence Address: PHARMACIA & UPJOHN, 7000 Portage Road, KZO-300-104, KALAMAZOO, MI, 49001, US

(73) Assignee: PHARMACIA ITALISA S.P.A.

(21) Appl. No.: 10/542,253

(22) PCT Filed: Jan. 30, 2004
(86) PCT No.: PCT/EP04/50058

§ 371 Date: Mar. 23, 2006

(30) Foreign Application Priority Data
Feb. 21, 2003 (IT) RM2005A000074

(51) Int. Cl. A61K 9/20

(52) U.S. Cl. 424/464

(57) ABSTRACT

The present invention relates to a pharmaceutical composition suitable for oral administration, in the form of semisolid matrix, comprising an active ingredient poorly soluble in water and present in a quantity of from 15 to 45% by weight of the percent composition of the pharmaceutical composition; a surfactant agent constituted by a polyglycolised glyceride; and a pharmaceutically acceptable hydrophilic carrier.

The present invention relates to semi-solid pharmaceutical formulations for immediate release, intended for the oral administration of drugs that are poorly soluble in water. The invention particularly relates to formulations with high percentage loading of active ingredient within the semi-solid matrix.

### BACKGROUND ART

One of the formulating approaches described in the technical-scientific and patent literature to improve the bio-pharmaceutical properties of active ingredients with poor solubility in water is represented by dispersions and solid solutions [W. L. Chiou et al, Journal of Pharmaceutical Sciences, 1971; 60 (9), 1281-1301].

Said formulations usually consist of the formation of an eutectic mixture between a water-soluble or water-dispersible excipient and the active ingredient. Said eutectic is formed by means of a preparatory technique of co-melting the active ingredient and the excipient or by using an organic solvent (solvent solubilisation and evaporation).

If said eutectic mixture is not formed, the dissolution rate of the active ingredient and hence, consequently, its potential improved oral bio-availability, are favoured by the dispersion of the hydrophobic active ingredient, poorly soluble in water, in the hydrophilic matrix constituted by the excipient, rather than by the partial solubilisation of the active ingredient in the excipient.

All effects mentioned above have an impact on the absorbability of the active ingredient through the gastrointestinal membranes, both because the dissolution rate is more rapid in contact with the biological fluids than the one achievable by conventional pharmaceutical forms such as tablets and capsules (since the dissolution rate is the limiting factor of absorbability of active ingredients for hydrophobic drugs, this entails a considerable bio-pharmaceutical advantage), and because the drug in excess administered (the quantity of drug that exceeds its solubility in the gastrointestinal fluids), precipitates out again as fine particles of colloidal dimensions, promoting even more the dissolution rate thanks to the significant increase of the surface area.

A second important advantage of solid dispersions is provided by the potential ease of preparation. For example, the development of technologies that allow to fill hard gelatine or hydroxypropylcellulose capsules with molten semi-solid mixtures allow to make the preparation of said formulating system simple and extremely reproducible even with respect to conventional oral solid forms [A. Serajuddin; Journal of Pharmaceutical Sciences, 1999; 88 (10), 1058-1066].

One of the intrinsic limits of this technology, however, is linked to the fact that it is very difficult to load within the semi-solid matrices high percentages (expressed as percent ratio between the weight of the active principle in the formulation and the total weight of the semi-solid formulation) of active ingredient without changing their dissolution properties and/or their workability. For example, as is readily apparent from a literature work by S. Dordunoo et al [Drug Development and Industrial Pharmacy; 1991; 17 (12), 1685-1713], the progressive increase of the quantity of poorly water-soluble drug (in the specific case Triamterene and Temazepam) in the hydrophilic matrix based on polyethylene glycol or Gelucire® 44/14 (Lauroil Macrogol-32 glyceride; Gattefossé), entails a significant reduction of the dissolution rate thereof.

For active ingredients requiring a significant loading inside the matrix (for instance, equal to or greater than 20% by weight) said semi-solid matrices prove to be ineffective in maintaining the properties of immediate release of the active ingredient, with a significant drop in dissolution velocity for weight concentrations exceeding 5% for triamterene and 15% for temazepam, thus influencing the availability of the drug to the absorption site. Above said concentrations, in the specific case the kinetics of release of the active ingredient from the formulation is governed by the intrinsic solubility of the active ingredient which is limited.

In addition, if an efficient loading is achieved, potential polymorph transformations of the excipient may influence, with the time and the ageing of the formulation, the release properties of the active ingredient, preventing the development of a formulation that maintains reproducible characteristics and bio-pharmaceutical properties [San Vicente et al., Proceedings of the 2nd World Meeting APGI/APV, Paris May 25-28, 1998, 261-262, W. Sutananta et al, International Journal of Pharmaceutics; 1994; 111, 51-62]

With very few exceptions, the above statements constitute the state of the art in the sector. For example, although Abdul Fatth et al. (International Journal of Pharmaceutics, 2002; 235, 17-23) describe a semisolid matrix of Halofantrine 40% loaded in a mixture of PEG800, polyvinyl pyrrolidone K30 or Gelucire® 44/14, this extremely simple formulation approach cannot be pursued for the majority of the active ingredients at the same concentrations obtaining an immediate release of the active ingredient from the formulation or, even more, having a formulation which, for its viscosity characteristics, can be produced in an effective and reproducible manner. For example, said experimental conditions are proven not to be viable for obtaining a formulation for immediate release of SU-6668 or of SU-14813, the molecules whereon the experimental data provided below are based.

Additionally, surfactant agents within the formulation are known to be used only for improving the properties of dissolution and subsequent absorption of the active ingredient through the gastrointestinal membranes and not to improve the loading properties of the active ingredient within the matrix itself. For example, the work by Khoo et al. [International Journal of Pharmaceutics; 2000; (205) 65-78], describes how the use of a surfactant substance like Vitamin E-TPGS within the formulation improves the characteristics of absorbability of the active ingredient by the formation of micro-emulsified systems in solution, but it has not a direct effect on the process of obtaining and producing the formulation itself.

Other examples in the literature where use of a surfactant substance to improve the properties of dissolution of the active ingredient is described, are the works published in the Journal of Pharmaceutical Sciences, 1999, 88 (10), 1058-1066 or in Drug Development and Industrial Pharmacy 1991, 17 (12); 1685-1713. In the latter publication, the addition of 2 or of 5% of polysorbate 80 (Tween 80) to a matrix of PEG 1500 containing 10% by weight of Triamterene, allows to release at least 80% of the active ingredient in an hour.

Tween 80 is also mentioned in another work to improve the release properties of a PEG 1000 matrix containing 15% by weight of active ingredient (Journal of Pharmaceutical Sciences, 79(5) 1990, 463-464).

### DETAILED DESCRIPTION OF THE INVENTION

It was surprisingly found that, in the case of poorly hydro-soluble active ingredients, the addition of a surfactant substance to a semisolid hydrophilic matrix allows not only to improve the dissolution properties of the active ingredient, but above all it allows to obtain a significant and unexpected increase in the ability to load the active ingredient into the matrix. These properties are very important in all cases where a high daily dosage is required. It was thus possible to obtain loading values of up to 30%, by weight, of the percent composition of the pharmaceutical formulation, maintaining unaltered the properties of active ingredient release from the matrix. In addition to obtaining the immediate release of the active principle, the formulation of the invention determines additional advantages, such as the considerable improvement of the workability properties of the active ingredients characterised by poor technological properties, such as a low apparent density, limited flowability and high toxic potential.

Yet more surprisingly, such effects are not obtained with any surfactant substance, but in particular using as a surfactant a polyglycolised glyceride.

Surfactants of this kind are not commonly used alone within the formulation, but are only employed in synergy with other surfactant substances (see for example International Journal of Pharmaceutics 1995, 118, 221-227, where the synergetic effect of Labrasol® and Tween 80 allows to obtain an improved oral bio-availability).

Therefore, a first object of the present invention is a pharmaceutical composition suitable for oral administration, in the form of semi-solid matrix, comprising: an active ingredient that is poorly soluble in water and present in a quantity of from 15 to 45% in weight of the percent composition of the pharmaceutical composition; a surfactant agent constituted by a polyglycolised glyceride; and a pharmaceutically acceptable hydrophilic carrier.

In a particular aspect of the invention, the active ingredient is present in quantities varying from 20 to 40% by weight of the percent composition of the pharmaceutical composition.

The term “active ingredient poorly soluble in water” in the present invention means a pharmacologically active agent characterised by a solubility in water that is equal to or lower than 0.1% in weight/volume ratio, i.e. to 1 mg/ml.

Preferably, the active ingredient of the invention is constituted by indolinone derivatives as described by Sugen Inc.—USA in several patents and patent applications, such as the U.S. Pat. Nos. 5,880,141 and 5,792,783 and the PCT patent application no. WO99/61422. Said compounds, being able to modulate the transduction of the mitogenic signal mediated by a tyrosin-kinase enzyme, are useful for therapeutic purposes to regulate, modulate and/or inhibit abnormal cell proliferation.

These compounds can be represented according to the following general formula (I) 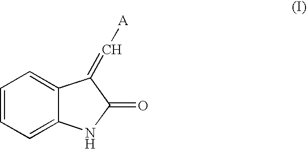
wherein A is a pyrrolic ring, optionally substituted in one or more positions with equal or different groups, selected among linear or branched lower alkyl, alkoxy, aryl, aryloxy, alkylaryl, alkoxyaryl, or groups —(CH₂)ₘCO₂H or —CONHR′, where m is 0 or an integer between 1 and 3 and R′ is a linear or branched lower alkyl, optionally substituted with one or more equal or different groups, selected among hydroxy, heterocyclyl, amine, alkylamine, dialkylamine; the indolinonic ring being optionally further substituted in one or more of the positions 4, 5, 6 and 7 with equal or different groups, selected among linear or branched lower alkyl, alkoxy, aryl, alkylaryl or alkoxyaryl.

In the present description, unless otherwise specified, the term heterocyclyl means a 5 or 6 term heterocyclic group with 1 to 3 hetero-atoms selected among O, N and S, such as morpholine, pyrrolydin, imidazolidin, piperidin and piperazin. The term lower alkyl means an alkyl group with 1 to 4 carbon atoms.

Particularly preferred are the pharmaceutical compositions where the active ingredient is selected among 3-[(3,5-dimethyl-1H-pyrrol-2-yl)methylene]-1,3-dihydro-2H-indol-2-one, also known as SU-5416; 5-[(1,2-dihydro-2-oxo-3H-indol-3-ylidene)methyl]-2,4-dimethyl-1H-pyrrol-3-propionic acid, also known as SU 6668; 3-{5-[6-(3-methoxy-phenyl)-2-oxo-1,2-dihydroindol-3-ylidenemethyl]-2-methyl-1H-pyrrol-4-yl}-propionic acid also known as SU-10994; 3-{5-methyl-2-[(2-oxo-1,2-dihydro-3H-indol-3-ylidene)methyl]-1H-pyrrol-3-yl}propionic acid, also known as SU-10944; 5-[(1,2-dihydro-5-fluoro-2-oxo-3H-indol-3-yliden)methyl]-N-[(2S)-2-hydroxy-3-morpholin-4-ylpropyl]-2,4-dimethyl-1H-pyrrol-3-carboxamide also known as SU-14813 and 5-[(1,2-dihydro-5-fluoro-2-oxo-3H-indol-3-yliden)methyl]-N-(2-diethylaminoethyl)-2,4-dimethyl-1H-pyrrol-3-carboxamide also known as SU-11248, represented by the following formulas:

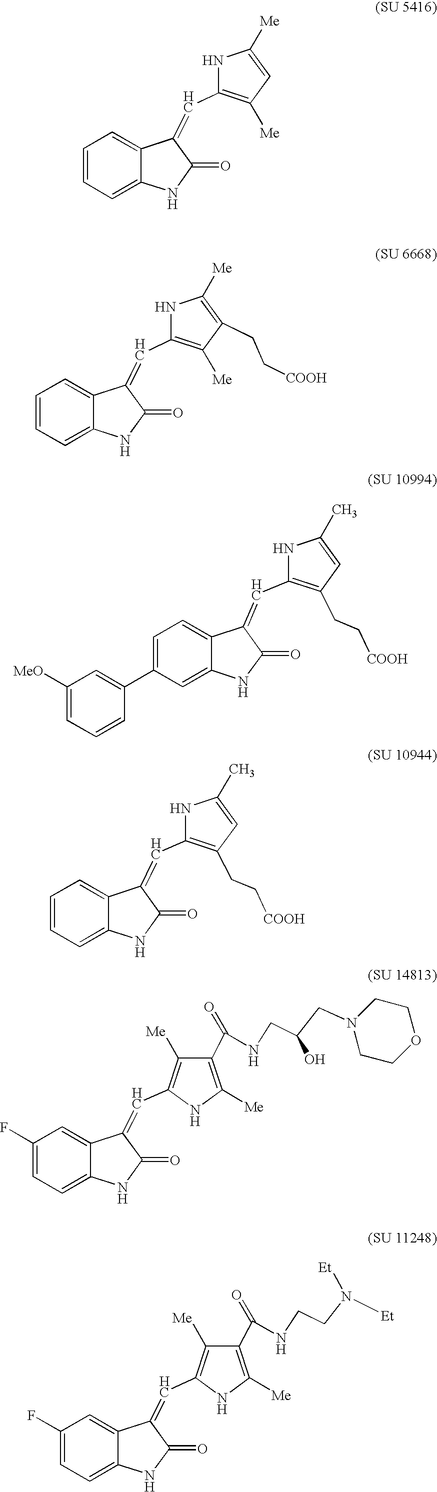

The pharmaceutically acceptable salts of the aforesaid compounds constitute an additional active principle, particularly preferred in the present invention. Among the latter can be mentioned, by way of example, SU-14813-L-Maleato.

In addition to the aforesaid compounds, the person skilled in the art will be able to apply the pharmaceutical composition of the invention also to other active ingredients characterised by poor solubility in an aqueous medium, which require high daily dosages. Among them one can mention, purely by way of non limiting example, anti-tumour antibiotics, such as anthracyclins; thymidilate-synthetase inhibitors, for instance capecitabin; inhibitors of the epidermal growth factor receptor; protease inhibitors (anti-HIV) such as amprenavir, indinavir, nelfinavir, ritronavir, squinavir or lopinavir; antimicrotubule agents included, for example, taxanes comprising paclitaxel and docetaxel vinca alkaloids; angiogenesis inhibitors like thalidomide; cycloxigenase-2 inhibitors such as celecoxib, valdecoxib, parecoxib and rofecoxib; aromatase inhibitors; alkylating agents, including estramustine phosphate; antimetabolites; hormonal agents like tamoxifen; platinum analogues including, for instance, cisplatinum, carboplatinum and oxalyplatinum and transcriptase inhibitors.

As stated, a characterising part of the present invention is the presence in the pharmaceutical composition of a surfactant agent constituted by a polyglycolised glyceride. The polyglycolised glycerides which can be used in the present invention are mixtures of known monoesters, diesters, and triesters in glycerol and known monoesters and diesters of polyethylene glycol with a mean relative molecular mass between about 300 and 6000. They may be saturated or unsaturated and can be obtained by the partial transesterification of triglycerides with polyethylene glycol or by esterification of glycerol and polyethylene glycol with fatty acids, using known reactions. Preferably, the fatty acids contain between 8 and 22 carbon atoms, particularly between 8 and 18 carbon atoms. Examples of natural vegetable oils which can be used are almond oil and palm oil.

The surfactant agent of the invention preferably has a high value of Hydrophilic-Lipophilic Balance, or HLB, in particular ranging between 4 and 14. The HLB scale is a numeric scale from 0 to 14, where the lower values denote lipophilic and hydrophobic substances whilst the higher values denote hydrophilic and lipophobic substances.

A particularly preferred saturated surfactant agent is caprylcaproyl macrogol-8-glyceride commercially available with the name Labrasol® (Gattefossè, Saint-Priest, France), which is liquid at room temperature and in which the predominant fatty acids are caprylic acid (C₈) and capryc acid (C₁₀).

Particularly preferred unsaturated surfactant ingredients are linoleyl macrogol-6-glyceride and Oleyl macrogol-6-glyceride, commercially available as Labrafil® M2125 and Labrafil® M1944 (Gattefossé, Saint Priest, France).

According to the present invention, the surfactant agent is present in the pharmaceutical composition in quantities that vary from about 2% to about 40% by weight, preferably from about 10% to about 30% by weight.

The pharmaceutical composition further comprises a pharmaceutically acceptable carrier forming the semisolid matrix. Said component, which constitutes an inert excipient, serves the function of favouring solubilisation and improving active ingredient release properties.

Therefore, the bio-availability of the active ingredient inside the body is improved and it can thus be effectively administered orally. Among the carriers, can be mentioned glycerides, medium and long chain fatty acids, hydrogenated and non hydrogenated polyoxyethylene vegetable oils and polymers with low melting point. The authors of the present invention have observed that the use, as carriers, of mixtures of polygycolised glycerides C₈-C₁₈ with a high value of HLB (see above), or of polymers with low melting point, particularly favour the diffusion through the membrane and the passage of the active ingredient into the blood. The polyglycolised glycerides most suitable as carriers are preferably saturated and with an HLB value of about 14. In particular, the saturated polyglycolised glyceride known by the trade name of Gelucire® 44/14 (Gattefossè) (Lauroyl Macrogol-32 glyceride) is used as a carrier according to the present invention. This substance is solid at room temperature, having a melting point of 44° C. and, appropriately combined with the surfactant agent, it contributes to form the semi-solid matrix in the characteristic form of the present pharmaceutical composition. In another particular embodiment of the invention the carrier employed is Poloxamer 188, a polymer commercially available with the name Lutrol® F68 (BASF, Germany) constituted by 81% of polyethylene glycol and 19% of polypropylene glycol and having a mean molecular weight of 8600.

The carrier is present in a proportion that varies from 30 to 90% by weight of the composition, and preferably between 40 and 70% by weight.

A preferred aspect of the present invention is a pharmaceutical composition suitable for oral administration, in the form of semisolid matrix, comprising SU-6668, Labrasol® and Gelucire® 44/14. An additional preferred aspect of the present invention is a pharmaceutical composition suitable for oral administration, in the form of semisolid matrix, comprising SU-14813, Labrasol® and Gelucire® 44/14. Yet another preferred aspect of the present invention is a pharmaceutical composition suitable for oral administration, in the form of semisolid matrix, comprising SU-14813, Lutrol® F68 and Labrasol®.

Other agents which may be added to the pharmaceutical composition of the invention are, for example, stabilising agents serving the purpose of maintaining the physical-chemical properties of the semi-solid matrix during the production and storage phases. Among these, the following chemical classes can be mentioned by way of example: lecithins; phospholipids; pharmaceutically acceptable oils, such as soy bean oil and the like.

Moreover, the semisolid matrix may contain other excipients such as an agent that favours dispersion and/or a surfactant and/or an agent that modifies viscosity and/or antioxidant and chelating agents and/or solubilising agents.

An agent that favours dispersion can be constituted by cellulose and its derivatives, such as carboxymethylcellulose and natural rubbers; a surfactant can comprise poloxamers, medium chain triglycerides, etoxilate esters, polyglycerol esters, alkylic polyoxiethylene ether, sorbitane esters; as viscosity modifying agent can be included hydrogenated and non hydrogenated vegetable oils, glycerol esters, polyglycerol esters and esters of propylene glycol; a solubilising agent can be constituted by ethanol, triacetin, propylene glycol or cyclodextrins.

Another aspect of the invention is constituted by a method for the preparation of the pharmaceutical composition, in the form of semisolid matrix, which comprises: dissolving or dispersing an indolinone derivative into the surfactant agent, in particular Labrasol®, to obtain a homogeneous and viscous mixture; adding, under stirring, the mixture thus obtained to the molten carrier, in particular Gelucire® 44/14, until obtaining a homogeneous mixture.

The mixture is maintained under stirring for up to 48 hours at controlled temperature, above the melting point of the carrier.

This formulation is particularly indicated for filling pharmaceutical capsule. Therefore, a further object of the invention is an oral formulation that comprises a capsule and, as a content, the pharmaceutical composition in semi-solid form as defined above. This oral formulation can take the form of a pharmaceutical capsule.

The present invention is particularly advantageous for the production of forms of solid oral dosage which can be prepared by means of known techniques. Technologies for filling capsules with a liquid preparation are well known. In particular, compositions containing fatty acids with a length of the carbon chain exceeding 8 are poured into the capsules as a hot molten mass.

Therefore, an additional object of the present invention is a capsule constituted by an external coating and by a content, where the content comprises the pharmaceutical composition of the invention in the form of semisolid matrix, as described above.

The outer coating of the capsule may be constituted by hard gelatine, hydroxypropylcellulose, amid or any pharmaceutically acceptable material for the preparation of capsules. Mixtures containing Labrasol® are readily dispersed when the capsule in which they are contained breaks up.

In a particular aspect, the oral formulation of the invention can be used for treating cancer.

The pharmaceutical composition can be administered to a mammal, including man, with a need for the therapeutic effects of the formulation of indolinone derivatives described in the invention. The capsules of the invention can thus be employed to treat many different types of cancer which comprise, by way of example, colon, breast, lung, prostate, pancreas, liver, stomach, brain, kidney, uterus, cervix, ovary, urinary tract cancer and melanoma. Although the examples that follow refer to the use of the specific compounds SU-6668 and SU-14813, the formulation approach is applicable to any other indolinone derivative and, more in general, to all molecules characterised by poor solubility in water and to be administered with a high daily dosage.

Therefore, the following example are provided with the aim of illustrating the invention but must in no way be considered as limiting the scope of the invention.

### EXAMPLE 1

Solid Dispersion Based on Gelucire® 44/14. (Formulation A)

An adequate quantity of Gelucire® 44/14 was melted at 60° C. under magnetic stirring. 6 mL of molten carrier were added to 0.6 g of SU 6668 and dispersed under magnetic stirring. After 4 hours of stirring, capsules of “0” format hard gelatine were manually filled with 0.5 mL of molten dispersion.

The final composition of the formulation A was as follows:

| Component | effective % p/p |
|---|---|
| SU 6668 | 10 |
| Gelucire ® 44/14 | 90 |

The titre, content of correlated substances and the dissolution profiles were checked.

The results of the following formulation are provided in the following table:

Percent titre and correlated substances content:

| | |
|---|---|
| Formulation A | Titre (% theoretical) 100 |
| Formulation A | % Correlated 0 |

The table that follows shows the dissolution profile for the Formulation A—Gelucire® 44/14 and 10% of SU 6668. The results are expressed as a percentage of active ingredient released by the formulation relative to the theoretical value, as a function of time.

| | SU-6668 released |
|---|---|
| Time (minutes) | (% of theoretical value) |
| 15 | 7 |
| 30 | 60 |
| 45 | 92 |

Based on the results obtained, it was possible to prepare a solid dispersion based on Gelucire®44/14 containing 10% of SU 6668, stable, homogeneous and with a dissolution profile that assures that more than 90% of the active profile is released by the formulation within 45 minutes.

### EXAMPLE 2

Solid Dispersion Based on Gelucire® 44/14. (Formulation B)

7.5 mL of Gelucire® 44/14 were melted at 60° C. under magnetic stirring. 1.5 g of SU 6668 were dispersed under magnetic stirring to the melted carrier.

After 4 hours of stirring, “0” format gelatine capsules were manually filled with 0.5 mL of melted dispersion.

The final composition of the formulation B was as follows:

| Component | Effective % p/p |
|---|---|
| SU 6668 | 17 |
| Gelucire ® 44/14 | 83 |

The titre, content of correlated substances and the dissolution profiles were checked.

The results of the following formulation are provided in the following table:

Percent titre and correlated substances content:

| | | |
|---|---|---|
| Formulation B | Titre (% theoretical) 95 | C.V. 9.1 (n = 2) |
| Formulation B | % Correlated 0 | |

The table that follows shows the dissolution profile for the Formulation B—Gelucire® 44/14 and 17% of SU 6668. The results are expressed as a percentage of active ingredient released by the formulation relative to the theoretical value, as a function of time.

| | SU-6668 released | |
|---|---|---|
| Time (minutes) | (% of theoretical value) | C.V. |
| 30 | 60 | 48 |
| 45 | 88 | 30 |

The loading into the matrix of Gelucire® 44/14 with a higher content of active ingredient led to mixing problems between the powder of SU 6668 and the molten carrier.

This in turn resulted in a poorly homogeneous matrix, as demonstrated by the values obtained both by the variation coefficient of the titre % and on the release profiles (as highlighted by the high values of the variation coefficient of the individual points). In any case the release of the active principle from the matrix fails to reach 90% within the first 45 minutes of the experiment.

### EXAMPLE 3

Solid Dispersion Based on Gelucire® 44/14 and Tween 80 (Formulation C)

1.5 g of SU 6668 were worked in a mortar with an amount of Tween 80; to the mixture were then added 7.5 mL of Gelucire® 44/14, previously melted at 60° C.

After 4 hours of stirring, “0” format gelatine capsules were filled manually with 0.5 mL of molten dispersion. Initially, a quantity of surfactant agent was added such as to assure a reduction of the apparent density of the active ingredient, sufficient to obtain a load in the matrix of 10% by weight.

The final composition of the formulation C was as follows:

| Component | effective % p/p |
|---|---|
| SU 6668 | 10 |
| Gelucire ® 44/14 | 71 |
| Tween80 | 19 |

The titre, content of correlated substances and the dissolution profiles were checked.

The results of the following formulation are provided in the following table:

Percent titre and correlated substances content:

| | |
|---|---|
| Formulation C | Titre (% theoretical) 100 |
| Formulation C | % Correlated 0 |

The table that follows shows the dissolution profile for the Formulation C—Gelucire® 44/14 and Tween 80 con 10% of SU 6668. The results are expressed as a percentage of active ingredient released by the formulation relative to the theoretical value, as a function of time.

| | SU-6668 released |
|---|---|
| Time (minutes) | (% of theoretical value) |
| 15 | 62 |
| 30 | 100 |
| 45 | 100 |

Working the SU 6668 with the Tween 80 allowed easily to load 10% of active principle within the matrix, obtaining a stable and homogeneous mixture from which the active principle is completely released during the dissolution experiment within 45 minutes.

### EXAMPLE 4

Solid Dispersion Based on Gelucire® 44/14 and Labrasol®(Formulation D)

1.5 g of SU 6668 were worked in a mortar with an amount of Labrasol®; the mixture was then added to 7.5 mL of Gelucire® 44/14, previously melted at 60° C.

After 4 hours of stirring, “0” format gelatine capsules were filled manually with 0.5 mL of molten dispersion.

A quantity of surfactant agent was added such as to assure a reduction of the apparent density of the active ingredient, sufficient to obtain a load in the matrix of 10% p/p.

The final composition of the formulation D was as follows:

| Component | effective % p/p |
|---|---|
| SU 6668 | 10 |
| Gelucire ® 44/14 | 71 |
| Labrasol ® | 19 |

The table that follows shows the dissolution profile for the Formulation D—Gelucire® 44/14 and Labrasol® with 10% of SU 6668. The results are expressed as a percentage of active ingredient released by the formulation relative to the theoretical value, as a function of time.

| | SU-6668 released |
|---|---|
| Time (minutes) | (% of theoretical value) |
| 15 | 72 |
| 30 | 100 |
| 45 | 100 |

Working the SU 6668 with the Labrasol® allowed easily to load 10% of active principle within the matrix, obtaining a stable and homogeneous mixture from which the active principle is completely released during the dissolution experiment within 45 minutes.

### EXAMPLE 5

Solid Dispersion Based on Gelucire® 44/14 and Tween 80 (Formulation E)

1.5 g of SU 6668 were worked in a mortar with an amount of Tween 80; the mixture was then added to 7.5 mL of Gelucire® 44/14, previously melted at 60° C.

After 4 hours of stirring, “0” format gelatine capsules were filled manually with 0.65 mL of molten dispersion. A quantity of surfactant agent was added such as to assure a reduction of the apparent density of the active ingredient, sufficient to obtain a load in the matrix of 14% p/p.

The final composition of the formulation E was as follows:

| Component | effective % p/p |
|---|---|
| SU 6668 | 14 |
| Gelucire ® 44/14 | 72 |
| Tween8O | 14 |

The titre, content of correlated substances and the dissolution profiles were checked.

The results of the following formulation are provided in the following table:

Percent titre and correlated substances content:

| | |
|---|---|
| Formulation E | Titre (% theoretical) 84 |
| Formulation E | % Correlated 0 |

The table that follows shows the dissolution profile for the Formulation E—Gelucire® 44/14 and Tween 80 with 14% of SU 6668. The results are expressed as a percentage of active ingredient released by the formulation relative to the theoretical value, as a function of time.

| | SU-6668 released |
|---|---|
| Time (minutes) | (% of theoretical value) |
| 15 | 30 |
| 30 | 72 |
| 45 | 80 |

Working the SU 6668 with the Tween 80 allowed to load 14% of active principle within the matrix with difficulty, with a less efficient (slower) profile of release of the active principle from the formulation than the results previously mentioned in Example 3.

### EXAMPLE 6

Solid Dispersion Based on Gelucire® 44/14 and Labrasol®(Formulation F)

1.5 g of SU 6668 were worked in a mortar with a quantity of Labrasol®; the mixture was then added to 7.5 mL of Gelucire® 44/14, previously melted at 60° C.

After 48 hours of stirring, “0” format gelatine capsules were filled manually with 0.65 mL of molten dispersion.

A quantity of surfactant agent was added such as to assure a reduction of the apparent density of the active ingredient, sufficient to obtain a load in the matrix of 15% p/p.

The final composition of the formulation F was as follows:

| Component | effective % p/p |
|---|---|
| SU 6668 | 15 |
| Gelucire ® 44/14 | 69 |
| TweenSO | 16 |

The titre, content of correlated substances and the dissolution profile were checked.

The results of the following formulation are set out below:

Percent titre and correlated substances content:

| | |
|---|---|
| Formulation F | Titre (% theoretical) 95 |
| Formulation F | % Correlated 0 |

The table that follows shows the dissolution profile for the Formulation G—Gelucire® 44/14 and Labrasol® with 15% of SU 6668. The results are expressed as a percentage of active ingredient released by the formulation relative to the theoretical value, as a function of time.

| | SU-6668 released |
|---|---|
| Time (minutes) | (% of theoretical value) |
| 15 | 60 |
| 30 | 94 |
| 45 | 100 |

Working the SU 6668 with the Labrasol® allowed easily to load 15% of active principle: the resulting mixture is stable and homogeneous.

The release profile and the dissolution rate of the active ingredient are faster than those described in Example 5.

### EXAMPLE 7

Solid Dispersion Based on Gelucire® 44/14 and Labrasol® (Formulation G)

5 g of SU 6668 were worked in a mortar with a quantity of Labrasol®; the mixture was then added to 4 mL of Gelucire® 44/14 melted at 60° C.

After 4 hours of stirring, “0” format gelatine capsules were filled manually with 0.65 mL of molten dispersion.

An amount of surfactant agent was added such as to assure a reduction of the apparent density of the active ingredient, sufficient to obtain a load in the matrix of 30% p/p.

The final composition of the formulation G was as follows:

| Component | effective % p/p |
|---|---|
| SU 6668 | 31 |
| Gelucire ® 44/14 | 43 |
| Labrasol ® | 26 |

The titre, content of correlated substances and the dissolution profile were checked.

The results of the following formulation are provided in the following table:

Percent titre and correlated substances content:

| | |
|---|---|
| Formulation G | Titre (% theoretical) 99 |
| Formulation G | % Correlated 0 |

The following table shows the dissolution profile for the formulation G—Gelucire® 44/14 and Labrasol® with 31% SU-6668. The results are expressed as the percentage of active ingredient released by the formulation relative to the theoretical value.

| | SU-6668 released |
|---|---|
| Time (minutes) | (% of theoretical value) |
| 15 | 15 |
| 30 | 67 |
| 45 | 92 |

Working the SU 6668 with Labrasol® allowed easily to obtain a matrix in Gelucire® 44/14 containing 31% of active ingredient.

Said matrix was easily worked, the values of percent titre are good to demonstrate a good chemical stability of the active ingredient and homogeneity of the suspension during the working phase.

The release profiles assure a greater than 90% release of the active ingredient within 45 minutes.

### EXAMPLE 8

Solid Dispersion Based on Poloxamer 188 and Labrasol®

An appropriate quantity of SU 14813-L-Maleate is worked in a mortar with Labrasol® with weight ratio 1:1 until obtaining a sufficient reduction of the density of the active ingredient power.

To this mixture is added Poloxamer 188 (Lutrol® F68, BASF, Germany), previously melted at 65° C., in a weight ratio 3:1 with the active ingredient.

Mixing is applied until obtaining a homogeneous distribution of the active ingredient in the matrix, then the mixture is placed in capsules of suitable dimensions for the required dosage.

| Component | effective % p/p |
|---|---|
| SU 14813-L-Maleate | 25 |
| Lutrol ® F68 | 50 |
| Labrasol ® | 25 |

### EXAMPLE 9

Solid Dispersion Based on Gelucire® 44/14 and Labrafil®

An appropriate quantity of SU 14813-L-Maleate is worked in a mortar with Labrafil® with weight ratio 2:1 until obtaining a sufficient reduction of the density of the active ingredient power.

To this mixture is added Gelucire® 44/14 previously melted at 60° C., in a weight ratio 3.5:1 with the active ingredient.

Mixing is applied until obtaining a homogeneous distribution of the active ingredient in the matrix, then the mixture is placed in capsules of suitable dimensions for the required dosage.

| Component | effective % p/p |
|---|---|
| SU 14813-L-Maleate | 25 |
| Gelucire ® 44/14 | 62.5 |
| Labrafil ® | 12.5 |

### EXAMPLE 10

Solid Dispersion Based on Gelucire® 44/14 and Labrasol®

An appropriate quantity of SU 14813-L-Maleato is worked in a mortar with Labrasol® with weight ratio 1:1 until obtaining a sufficient reduction of the density of the active ingredient power.

To this mixture is added Gelucire® 44/14 previously melted at 60° C., in a weight ratio 3:1 with the active ingredient. Mixing is applied until obtaining a homogeneous distribution of the active ingredient in the matrix, then the mixture is placed in capsules of suitable dimensions for the required dosage.

| Component | effective % p/p |
|---|---|
| SU 14813-L-Maleate | 20 |
| Gelucire ® 44/14 | 60 |
| Labrasol ® | 20 |

### EXAMPLE 11

Solid Dispersion Based on Poloxamer 188 and Labrasol®

An appropriate quantity of SU 14813-L-Maleate is worked in a mortar with Labrasol® with weight ratio 2:1 until obtaining a sufficient reduction of the density of the active ingredient power.

To this mixture is added Poloxamer 188 (Lutrol® F68, BASF, Germany) previously melted at 60° C., in a weight ratio 3:1 with the active ingredient.

Mixing is applied until obtaining a homogeneous distribution of the active ingredient in the matrix, then the mixture is placed in capsules of suitable dimensions for the required dosage.

| Component | effective % p/p |
|---|---|
| SU 14813-L-Maleate | 20 |
| Lutrol ® F68 | 60 |
| Labrasol ® | 10 |

1. A pharmaceutical composition suitable for oral administration, in the form of semisolid matrix, comprising:
an active ingredient poorly soluble in water and present in a quantity of from 15 to 45% by weight of the percent composition of the pharmaceutical composition; a surfactant agent constituted by a polyglycolised glyceride; and a pharmaceutically acceptable hydrophilic carrier.

2. A composition according to claim 1, wherein the active ingredient is present in a quantity of from 20 to 40% by weight of the percent composition of the pharmaceutical composition.

3. A composition according to claims 1 or 2, wherein the active ingredient is constituted by an indolinone derivative represented by the general formula (I) 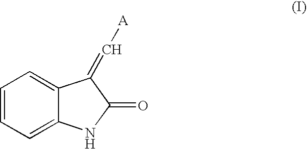
wherein A is a pyrrolic ring, optionally substituted in one or more positions with equal or different groups, selected among linear or branched lower alkyl, alkoxy, aryl, aryloxy, alkylaryl, alkoxyaryl, or groups —(CH₂)ₘCO₂H or —CONHR′, where m is 0 or an integer between 1 and 3 and R′is a linear or branched lower alkyl, optionally substituted with one or more equal or different groups, selected among hydroxy, heterocyclyl, amine, alkylamine, dialkylamine; the indolinonic ring being optionally further substituted in one or more of the positions 4, 5, 6 and 7 with equal or different groups, selected among linear or branched lower alkyl, alkoxy, aryl, alkylaryl or alkoxyaryl.

5. A pharmaceutical composition according to claim 3, wherein the indolinone derivative is selected from the group consisting of SU 5416, SU 6668, SU 10944, SU 10994, SU 14813, SU 11248 and the respective pharmaceutically acceptable salt forms.

6. A composition according to any one of claims 1 to 4, wherein the surfactant agent is selected from the group consisting of Labrasol®, Labrafil® M2125 and Labrafil® M1944.

7. A composition according to any one of claims 1 to 5, wherein the carrier is a saturated polyglycolised glyceride or a polymer with low melting point.

8. A composition according to claim 6 wherein the carrier is selected between Gelucire® 44/14 and Lutrol® F68.

9. A composition according to any one of claims 1 to 7, comprising SU-6668, Labrasol® and Gelucire® 44/14.

10. A composition according to any one of claims 1 to 7, comprising SU-14813, Labrasol® and Gelucire® 44/14

11. A composition according to any one of claims 1 to 7, comprising SU-14813, Lutrol® F68 and Labrasol®.

12. A composition according to any one of claims 1 to 10 further comprising an agent that favours dispersion and/or a surfactant and/or an agent that modifies viscosity and/or antioxidant and chelating agents and/or solubilising agents.

13. An oral formulation comprising a capsule and, as its content, the pharmaceutical composition in semi-solid form as defined in any one of claims 1 to 11.

14. An oral formulation according to claim 12 for use in the treatment cancer.

15. Use of an indolinone derivative according to claim 3, a surfactant agent consisting of a polyglycolised glyceride and a pharmaceutically acceptable hydrophilic carrier, in the preparation of a medicament intended for oral administration in the treatment of cancer.

16. Use of Labrasol®, Labrafil® M2125 or Labrafil® M1944 as surfactant agents in a pharmaceutical composition comprising an indolinone derivative according to claim 3 and a pharmaceutically acceptable hydrophilic carrier.

17. Use of Labrasol® as surfactant agent in a pharmaceutical composition comprising an indolinone derivative according to claim 3 and Gelucire® 44/14.

18. Use of Labrasol® as surfactant agent in a pharmaceutical composition comprising SU 6668 and Gelucire® 44/14.

19. A method for preparing the pharmaceutical composition according to claim 3, comprising: dissolving or dispersing the indolinone derivative in the surfactant agent, until obtaining a homogeneous and viscous mixture; and adding under stirring the mixture thus obtained to the molten carrier until obtaining a homogeneous mixture.
